(19) United States
(12) Patent Application Publication
(10) Pub. No.: US 2003/0091642 A1
(43) Pub. Date: May 15, 2003

(54) Composition in the form of a gel for receiving an active ingredient in a solution or suspension, especially for application on a mucous membrane and method of production thereof

(76) Inventor: Jack Auzerie, Sainte-Eulalie (FR)

Correspondence Address: YOUNG & THOMPSON, 745 SOUTH 23RD STREET 2ND FLOOR, ARLINGTON, VA, 22202

(21) Appl. No.: 10/149,695

(22) PCT Filed: Dec. 14, 2000
(86) PCT No.: PCT/FR00/03533

(30) Foreign Application Priority Data
Dec. 14, 1999 (FR) 99/15763

(51) Int. Cl. A61K 9/14

(52) U.S. Cl. 424/486; 424/488

(57) ABSTRACT

A composition in the form of a gel provided for the treatment of mucosa with an active principle, particularly the vaginal mucosa, includes at least one thermoreversible gel, at least one bioadhesive product, and at least one active principle in solution or suspension.

The present invention relates to a composition in the form of a gel for diffusion of an active principle and provided to be applied to a mucous membrane and more particularly for use in vaginal treatments.

The invention also covers the process of developing this composition to obtain a suitable product.

The problem with treatment of sensitive regions such as the mucosa is a problem of application and maintaining the active principles on the region for a sufficient time to ensure the action of these principles, without giving rise to difficulty for the person who is treated. Moreover, to be distributed commercially, it is necessary that the product be packaged in multi-doses or in single doses and be easy to use on the region because it is difficult of access.

This is the case with the vaginal route, which is for example given to explain the characteristics and advantages of the present invention, without the choice being limited to this single mucous membrane. Different medications could be combined with the gel for topical application. A first application could be antifungal.

It is known that micoses have a high tendency to develop at present for several reasons and particularly because it is impossible to prevent this by preliminary treatment because of factors favoring them. These micoses are for the most part opportunistic, iatrogenic and survive on a region weakened from modern therapies permitting satisfactory action in other regions but which give rise to these secondary effects. They also survive following stays in hospital localities.

There are known effective treatments for these micoses, but which remain above all localized with compounds which are essentially polyenic derivatives and azole or imidazole derivatives.

Such molecules are presented in solid form: tablets, pessaries or capsules.

Tablets have a diffuse action which do not permit a targeted action.

Pessaries such as vaginal capsules have a local action but limited diffusion. Moreover, the acceptability remains low and the results are less homogeneous and difficult to reproduce.

There are also known gels, but these, in contact with the mucosa, remain in gel form and have the tendency to loose their viscosity, which leads to flowing which is uncomfortable for the patient. Moreover, retention remains low, which limits the action of the active principles and/or require a longer treatment. Moreover, the packaging of the only known commercial product for the treatment of vaginal micoses is of the multi-dose type with a graduated applicator which is not particularly satisfactory for this type of pathology.

A second application could be antibiotics for curative purposes against vaginal bacteria, for example econazole and/or metronidazole or an antibiotic of the macrolide type.

A very pronounced increase in viscosity cannot be foreseen for problems of complexity of application to mucosa that are difficult of access.

This is why the problem is not limited to use in vaginal treatments but relates more generally to all the mucosa difficult of access, such as rectal, nasal, gingival or jugal mucosa.

The present invention provides a composition which permits maintaining ease of application of the gels, which ensures good impregnation of the treated mucosa, which has a high remanent strength, which is comfortable for the patient and which permits diffusion of a large ranges of active principles. Thus, in addition to the therapeutic products indicated above, all the products for which a local effect is sought, are adapted to be incorporated in a gel according to the present invention. There can be cited decongestant products, anti-inflammatory products, anti-allergenic products, antalgic products or anti-prurigenic products, substitutional or natural anti-conceptional hormonal products such as oestroprogestatives or synthetic anti-conception drugs such as danazol.

Thus the active principle used is incorporated in a thermo-reversible bioadhesive gel.

The characteristic of this gel, of being liquid at ambient temperature or at least of very low viscosity, permits diffusion by different means, particularly an aerosol, by packaging in a can with a propulsive gas, or with the help of a pocket valve. This use is carried out under the control of medical personnel. The lesioned regions are thus reached with precision and the gel, because of its bioadhesivity, remains on the region sufficiently long that the medical principle takes effect.

The prior art describes thermoreversible gels, particularly in U.S. Pat. No. 4,188,373 and Canadian patent 1,072,413.

In these patents, there are used polymers of polyoxyethylene-polyoxypropylene. These gels are used as vectors for medications to be deposited on the mucosa.

Thus targeted applications have been developed in ophthalmology, with different proprietary gels, described in U.S. Pat. Nos. 4,474,761 and 4,692,454.

European patent application 0 551 626 discloses thermo-rheological modifications of gels but without achieving the solutions claimed in the present invention. Moreover, this prior art does not focus on the applications particularly envisaged and on the possibilities of administration to patients.

The present invention provides a preparation adapted to be sprayed at ambient temperature for a better targeted application and having a strongly bioadhesive characteristic on the mucosa.

The different accompanying figures permit showing the curves used during the description which will follow, of a composition suitable for a particular field, but in a non-limiting manner.

The different figures show:

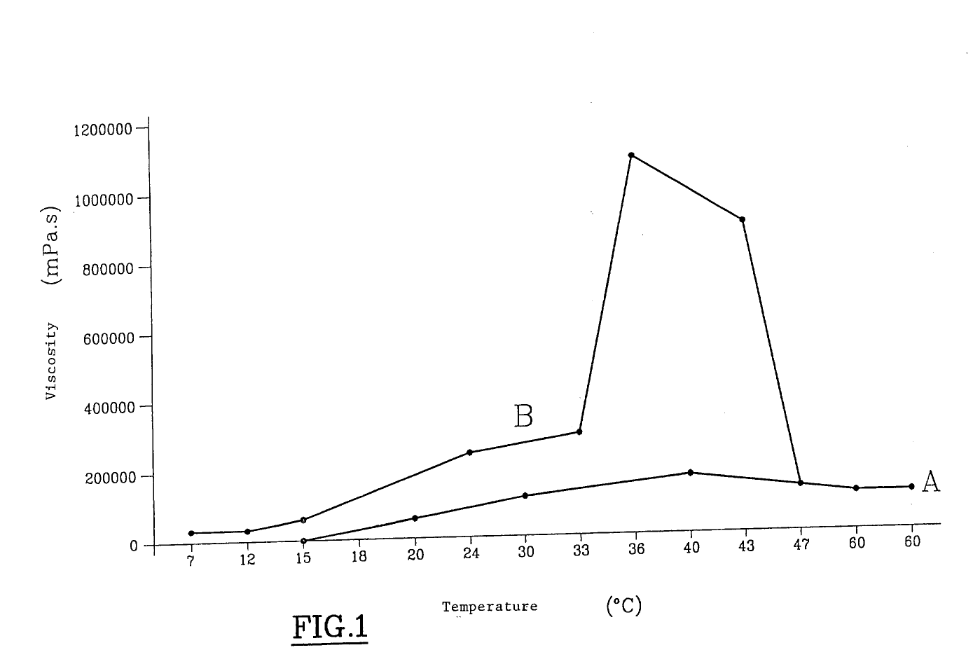

FIG. 1, a view of a curve of viscosity of a preferred composition of the invention, compared with the base product, and

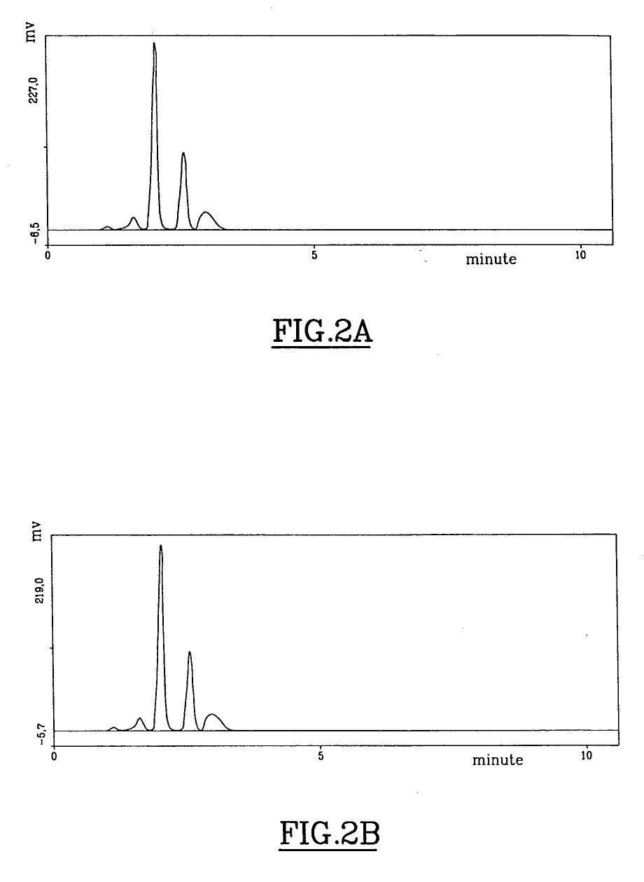

FIGS. 2A and 2B, chromatograms comparing a same composition immediately after production and after the passage of one month warmed to 45° C.

The general composition comprises the combination of a thermoreversible gel, a bioadhesive gel and at least one active principle.

The thermoreversible characteristic permits imparting to the gel a very low viscosity permitting it to be considered as a liquid at ambient temperature and viscous at body temperature. The liquid form before application facilitates the regular and reproducible distribution on the mucosa whilst the more viscous form permits better adherence to the mucosa by limiting flow.

The bioadhesive character permits improving the contact between the active principle and the mucosa, which increases the therapeutic effectiveness and the retention of the action so as to limit the number of applications and the duration of the treatment by improving observance. Similarly, the bioadhesive character reinforces the effect of viscosity arising from thermoreversibility of the gel and further limits possible flow to the extent of suppressing it in most cases.

In the treatment of vaginal micoses, the composition according to the invention, particularly suitable, is indicated hereafter:

15% Poloxamer: solubilizing, thickening, thermoreversibly gelling. There can be cited the family of poloxamers, which is to say copolymers of ethylene oxides and of propylene, which is sold under the name Lutrol F127.

0.5% Carbomer: bioadhesive gel. An example of commercial product satisfying this family of polymers of acrylic acid of high molecular weight is Carbopol 5904 whose viscosity is between 25,000 and 45,000 centipoise.

1% of active principle, in this case micronized econazol nitrate <50 μm, which is a known antimycosal.

preservation agents such as methyl parahydroxybenzoate and propyl parahydroxybenzoate, containing a sodium in the amount respectively of 0.1 and 0.05%,

0.2% sodium hydroxide, for neutralizing the solution, and

qsp distilled water used as a solvent (in a sufficient quantity to make up 100%) because it dissolves well in water contrary to the others such as butylhydroxyanisol also tested for the formulation.

Formulation is carried out in the following manner:

premixture 1; addition of portion of the water to the sodium hydroxide

premixture 2; dispersion of the active principle in the volume of water brought to a temperature of the order of 30 to 35° C., and

mixture with the defloculent of the poloxamer, of the carbomer and of premixtures 1 and 2.

There is obtained a white gel, thermoreversible and homogeneous with a pH of the order of 5.5, ready for packaging.

It will also be noted that in the formulation, the order of introduction has a certain importance because, preferably, the active principle is incorporated last, after the poloxamer. This latter has solubilizing properties which decrease the propensity of the active principle to form aggregates of particles and hence to give rise to inappropriate sedimentation.

FIG. 1 shows the curve of the comparative results obtained relating to variations of viscosity as a function of temperature, variations which are very significant. Smoothed curve A is the image of the variations of viscosity of Lutrol F127 at 25% alone, and curve B is the image of the abrupt increase of the viscosity at 33° C. of this same Lutrol in associated with Carbopol 5984.

It will be noted that the Lutrol used alone does not really have a thermogelling behavior.

The quantities of poloxamer adapted to produce the sought effects are comprised between 1.0% and 40.0%, more particularly between 5.0% and 20.0%.

The quantities of carbomer adapted to produce the sought effects in association with the poloxamer in the quantities indicated above, are comprised between 0.1% and 2.0%, more particularly between 0.5% and 1.0%.

Another series of comparative tests consists in carrying out tests on rabbit eye.

There is prepared:

a control composition based on Lutrol F127 at 25%, containing 0.01% of fluorescein. The composition has a viscosity of 20,000 centipoise at 30° C.

a test composition based on Lutrol 127 at 5% also containing 0.1% of fluorescein but with in addition 0.5% of Carbopol 5984. The composition also has a viscosity of 20,000 centipoise at 30° C.

The compositions are applied to the eye of five rabbits, the control preparation on the right eye and the test preparation on the left eye.

Retention is measured by the presence of fluorescein, by examining the fluorescence of the eyes of the rabbits subjected to UV illumination (254 nm).

The results indicate better retention of the fluorescein at the surface for the test composition:

| Rabbit | No. 1 | No. 2 | No. 3 | No. 4 | No. 5 |
|---|---|---|---|---|---|
| Control (Duration in hours) | 6 h | 4 h | 3 h | 2 h | 3 h |
| Test (Duration in hours) | 9 h | 12 h | 12 h | 10 h | 18 h |

Thus, the increase in retention is very significant because there exists a ratio of 3 to 5 if the extremes are eliminated.

Tests have also shown the interest of a possible addition of a cellulosic derivative such as monocrystalline cellulose sold under the name “Avicel”, a thixotropic agent, or preferably hydroxypropyl methylcellulose sold under the name “Methocel E5 premium”. Such a cellulosic derivative is synergetic with the poloxamer and permits reducing the range of transition and phase temperatures and increasing very substantially the viscosity of the gel during the thermogelling phase, in the course of exposure to heat.

Suitable quantities vary from 1 to 5% without the limits being barriers to the use but larger quantities do not much modify the properties whilst they can on the contrary cause the thermoreversible effect to disappear.

Moreover, one should not use a gel which is too viscous at ambient temperature, because non-fluidity can hinder the application to the mucosa on the one hand and such product is difficult to free from bubbles after passage through the dynamic mixing apparatus on the other hand.

According to the preferred composition, shelf life is of the order of 5 years in a stable manner, which is to say that the active principle remains in dispersed form or dissolved in the gel.

The curves of FIGS. 2A and 2B show this stability.

The description which will be given covers use for treatments of the vaginal mucosa afflictions, but the same is true for other mucosa of difficult access. The essence of the invention remains the combination of a poloxamer type gel and a product having bioadhesive characteristics, in the compositions and concentrations described, ensuring maximum bioadhesivity.

As to the active principles adapted to be associated with this composition, they can be of different natures and even be associated within a same composition. There can also be used antifungals, antibiotics or substitute hormonal or natural birth control products such as estroprogestatives or synthetic birth control preparations such as danazol.

Similarly, there can be cited decongestants, anti-inflammatories, anti-allergical, antalgic or anti-pruriginic compositions.

There can also be noted important results and particularly advantageous results from the compositions which have been indicated.

Low viscosity at ambient temperature permits recourse to particularly suitable administration means and which totally use the low viscosity at ambient temperature and strong bioadhesivity.

Under medical control, for example during a gynecological examination with the help of a speculum, the product can be vaporized or projected by the doctor in jets against the mucosa to be treated.

If the medication to be administered is an antifungal, the gynecologist can also apply the product directly to the infected region or regions.

The product adhering to the mucosa is of a concentration greater than the minimal inhibitory concentration for a sufficient time to eradicate the vectors responsible for the infection.

There can be used the same approach with an antibiotic treating unspecific vaginal conditions.

There can also be provided a multi-perforate device for introduction into a natural cavity to be treated (vagina, rectum . . . ), having an anatomical shape, the product being dispensed through this device by means of a dosing valve or a propellant gas.

There can also be self-administration.

The same advantages of the composition according to the invention result from low viscosity upon application and high retention once applied.

1. Composition in the form of a gel for the treatment of mucosa with an active principle, particularly vaginal mucosa, characterized in that it comprises in combination:
a poloxamer Lutrol F 127
a bioadhesive product Carbopol 5984, and
at least one active principle in solution or in suspension.

2. Composition according to any one of the preceding claims, characterized in that the active principle is an antifungal, an antiseptic, an antibiotic, a contraceptive or a combination of two or more of them.

3. Composition according to one of claims 1 or 2, characterized in that a cellulosic derivative is added to the composition.

4. Composition according to claim 3, characterized in that the cellulosic derivative is hydroxypropyl methylcellulose.

5. Composition according to any one of the preceding claims, characterized in that it comprises water as an excipient.

6. Composition according to any one of the preceding claims, characterized in that it comprises at least one preservative and neutralizer.

7. Composition according to claim 6, characterized in that the preservative is selected from sodium methyl parahydrobenzoate or sodium propyl parahydrobenzoate.

8. Composition according to claim 6 or 7, characterized in that the neutralizer is sodium hydroxide.

9. Process for the production of the composition according to any one of claims 1 to 8, characterized in that it consists in carrying out the sequence of the following steps:
premixture 1; addition of a portion of the excipient to the neutralizer,
premixture 2; dispersion of the active principle in a volume of excipient at a temperature of the order of 30 to 35° C., and
mechanical mixture of the thermoreversible gel, of the bioadhesive product and of premixtures 1 and 2.
